(19) United States
(12) Patent Application Publication
(10) Pub. No.: US 2006/0134265 A1
(43) Pub. Date: Jun. 22, 2006

(54) Method of producing organic material and use of the same

(76) Inventor: Vivian Godfrey Beukes, George (ZA)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/518,096

(22) PCT Filed: Jun. 25, 2003
(86) PCT No.: PCT/ZA03/00085

§ 371 Date: Jul. 28, 2005

(30) Foreign Application Priority Data
Jun. 25, 2002 (ZA) 2002/5092

(51) Int. Cl. A23K 1/18; C12P 1/00

(52) U.S. Cl. 426/2; 435/41

(57) ABSTRACT

The invention relates to a method of producing an organic material, which multiplies through a process of germination, wherein the method is aimed at increasing growth of the material and reducing the period necessary for harvesting the same. In particular, the method, which includes a two-stage fermentation process, comprises the steps of preparing a starter nutrient medium; adding a starter culture of an organic bacterial fungus to the nutrient medium; permitting the mixture of nutrient medium and fungus culture to undergo a first stage fermentation process; transferring the mixture to a fermentation container; and allowing the mixture to undergo a second stage fermentation process until the organic material has germinated fully. The invention also extends to the use of this material in a number of applications, and in particular in fire extinguishing, fire prevention applications and as a general thermal barrier.

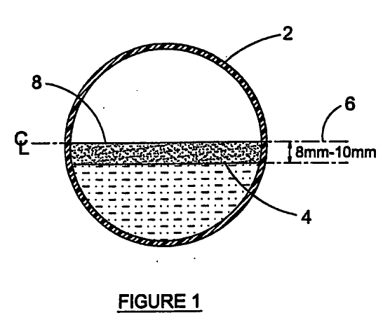

### TECHNICAL FIELD

The invention relates to a method of producing an organic material, which multiplies through a process of germination, wherein the method is aimed at increasing growth of the material and reducing the period necessary for harvesting the same. The invention also extends to the use of this material in a number of applications, in particular in fire extinguishing, fire prevention applications and as a general thermal barrier.

### BACKGROUND ART

Communalities of yeasts and bacteria have been known and applied for years inter alia in the preparation of fermented drinks and foodstuffs. One example of this symbiosis of bacteria and yeasts is the tea-fungus “Kombucha”, which has been used since as early as 1914 to cure ailments and illnesses such as stomach-intestine activity, haemorrhoids, joint rheumatism, constipation, arteriosclerosis, headaches, dizziness, high blood pressure, anxiety, dizziness and many other symptoms. In the preparation of Kombucha, a nutrient medium is prepared and a mushroom-type fungus material is allowed to grow on the nutrient medium. After a period of time, the fungus material is removed from the nutrient medium and discarded and the nutrient medium, which then contains various added elements, is used as a health drink.

However, the applicant's invention is focused on the previously discarded fungus-material. The applicant has found that the fungus-material has substantial heat-resistant properties and as such is capable of being used as a fire-retardant thermal barrier and indeed fire-extinguishing material. In addition, the applicant has developed a method of increasing growth of this fungus-type material and reducing the period necessary for harvesting the same.

### OBJECT OF THE INVENTION

It is accordingly an object of the present invention to provide a method of producing an organic material, wherein the method is aimed at increasing growth of the material and reducing the period necessary for harvesting the same.

It is a further object of the invention to provide for the use the organic material in a variety of different applications.

### DISCLOSURE OF THE INVENTION

A method of producing an organic material wherein the material multiplies through a process of germination and wherein the method is characterised therein that it includes a two-stage fermentation process, the method comprising the steps of preparing a starter nutrient medium in which an organic bacterial fungus will grow; adding a starter culture of the organic bacterial fungus to the nutrient medium; permitting the mixture of nutrient medium and fungus culture to undergo a first stage fermentation process; transferring the mixture to a fermentation container; and allowing the mixture to undergo a second stage fermentation process until the organic material has germinated fully.

The organic material may be a gelatinoid material in the form of a substantially flat sheet. The organic sheet material may be characterised therein that it first spreads over the surface of the nutrient medium and then thickens once the surface of the nutrient medium is covered. Once the material has thickened into the flat sheet, it is substantially self-supporting and capable of being removed from the nutrient medium.

The nutrient medium may be an infusion of water and plant material. Particularly, the nutrient medium may be an infusion of tealeaves and water. More particularly, the tealeaves may be selected from a group including, although not necessarily limited to, Cyclopia Intermedia, Matricaria Recutita, Aspalathus linearis and/or Lavender.

The organic bacterial fungus may be a fungus colony of the specific plant material to be infused during preparation of the nutrient medium. So, for example, if the nutrient medium is prepared from an infusion of Cyclopia Intermedia leaves in water, the fungus starter culture may be a fungus colony of Cyclopia Intermedia.

The method may include the further step of introducing an acidic medium into the starter nutrient medium for reducing pH of the same. In one form of the invention, the method includes the step of introducing distilled vinegar into the starter nutrient medium.

During fermentation, pH of the nutrient medium decreases and the starter nutrient medium becomes increasingly acidic the longer the fermentation process is allowed to occur. In fact, the nutrient medium may have a pH as low as 2.5 to 3.5 upon harvesting of the sheet material. The applicant has found that equally good results are achieved when the acidic nutrient medium from a previous fermentation process is introduced into the starter nutrient medium, as when distilled vinegar is used. Accordingly, in an alternative form of the invention, the method may include the step of introducing acidic nutrient medium from a previous fermentation process into the starter nutrient medium.

The first stage fermentation process may be for a period of between 3 and 5 days. The first stage fermentation process may be characterised therein that the mixture is not disturbed at all (e.g. touched, stirred, shaken, moved or otherwise displaced) during the first stage fermentation process.

During the first stage fermentation process the nutrient medium may be maintained at a temperature ranging between 20° C. and 30° C. Optimally, the nutrient medium is maintained at a temperature range of 23° C.-28° C.

The first stage fermentation process is executed in the absence of any direct sunlight.

The second stage fermentation process may be for a period of between 10 and 12 days. More particularly, the second stage fermentation process may be allowed to continue until the sheet material has grown to a thickness of between 8 mm and 10 mm.

During the second stage fermentation process the nutrient medium is again maintained at a temperature range of between 20° C. and 30° C., and optimally at a temperature range of 23° C.-28° C.

The fermentation container may be an elongate and substantially cylindrical container. For the purpose of this document, “cylindrical” will be interpreted to include a container having a circular, oval, ecliptic, square, triangular, rectangular, hexagonal or the like cross-section. The mixture may be introduced into the fermentation container such that the surface area of the nutrient medium is below the horizontal centerline of the cylindrical fermentation container. More particularly, the surface area of the nutrient medium may be between 8 mm and 10 mm below the horizontal centerline of the cylindrical fermentation container, the arrangement being such that the sheet material is permitted to germinate until it has reached the horizontal centerline of the fermentation container, after which it is removed, at which point the sheet material should have a thickness of between 8 mm and 10 mm.

In one form of the invention the fermentation container may be a fermentation pipe having a diameter in the order of 100 mm. The fermentation pipe may vary in length, but the applicant has found that a pipe length in the order of 6 m provides for good airflow and temperature control of the nutrient medium.

According to a second aspect of the invention there is provided a method of producing an organic material wherein the material multiplies through a process of germination, the method comprising the steps of preparing a starter nutrient medium in which an organic bacterial fungus will grow; adding an acidic medium to the starter nutrient medium for reducing pH of the same; adding a starter culture of the organic bacterial fungus to the nutrient medium; and permitting the mixture of acidic starter nutrient medium and fungus culture to undergo fermentation until the organic material has germinated fully.

The acidic medium may be distilled vinegar. Alternatively, the acidic medium may be an acidic nutrient medium from a previous fermentation process.

The method may be characterised therein that it includes a two-stage fermentation process wherein the mixture of starter nutrient medium and fungus culture is permitted to undergo a first stage fermentation process, after which the mixture is transferred to a fermentation container and allowed to undergo a second stage fermentation process until the organic material has germinated fully.

The method may include the further step of, subsequent to germination and harvesting of the organic sheet material, utilizing the then nutrient medium resulting from the fermentation process as the starter nutrient medium for growing a second organic sheet material, this further step being characterised therein that it does not require addition of a starter culture of the organic bacterial fungus to the nutrient medium. Depending on prevailing fermentation conditions, such as temperature and airflow, the process of harvesting the organic sheet material and utilizing the then nutrient medium resulting from the fermentation process as the starter nutrient medium for growing another organic sheet material, without the need for adding additional starter culture to the nutrient medium, may be repeated a number of times.

The material may be dried to form a dry sheet or pulverised to form a gel. The material may be characterised therein that it is non-toxic and biodegradable. The material further may be characterised therein that it is substantially self-adherent to most surfaces and as such can be sprayed onto surfaces when it is in the gel form.

The material may also be characterised therein that it comprises fire retardant properties.

The invention extends to the use of the material produced according to the invention as a fire extinguishing material for use, for example in commercial fire extinguishers, sprinkler systems for buildings, ships, trains or the like, in heat protective clothing, for use in extinguishing forest fires or vegetation fires.

The invention also extends to the use of the material produced according to the invention as a fire prevention material, for example as a lining material in buildings, aircraft, ships or the like, in paints, varnishes or the like to be applied to buildings, aircraft, ships or the like, in the manufacturing of roof tiles, dry walling, partitions, ceiling boards, cement formulations, on aircraft runway surfaces to decrease fire risks during emergency landings, inclusion in furniture upholstery, and the like.

The applicants have found that the material floats upon flammable liquid solvent such as petrol. Accordingly, the invention extends to the use of the material according to the invention for extinguishing solvent fires, such as petrol fires by spraying the material onto the fire.

The invention also extends to the use of the material according to the invention for emulsifying oil and water by introducing the material into the oil and/or water. More particularly, the invention extends to the use of the material in emulsifying oil and water during a process of recovering oil from a source, such as during oil winning from an oil well.

The invention further extends to the use of the material according to the invention for terminating smoke emission during a fire by spraying the material over the smoke, the material being characterised therein that it adheres to the smoke particles, increasing its particle weight and as such forcing it the ground.

### SPECIFIC EMBODIMENT OF THE INVENTION

Without limiting the scope thereof, the invention will now further be illustrated and exemplified with reference to the accompanying examples and the drawing, which is a transverse cross-sectional view through a fermentation pipe used by the applicant in the method according to the invention.

### EXAMPLE 1

A nutrient medium was prepared by adding 2 teaspoons of dried tealeaves to 1 litre of boiling water. The infusion was allowed to stand for approximately 15 minutes, after which the tealeaves were strained off. Subsequently, 70-100 grams of refined white sugar was added to the infusion while stirring the same to facilitate solution of the sugar. The liquid was allowed to cool down to between 20° C. and 25° C., after which 10V/O fungus colony was added to the same. If the temperature is too high the fungus colony will die.

The liquid was transferred to a container for the first stage fermentation process and left for 4 weeks. During the first stage fermentation process the liquid nutrient medium was maintained at 23° C.-27° C.

After 4 weeks, the liquid was removed to elongate plastic fermentation pipes (2) for the second stage fermentation process. The pipes (2) were filled so that the surface area of the nutrient medium (4) was between 8 mm and 10 mm below the horizontal centreline (6) of the pipes (2), as illustrated in the accompanying drawing. The pipes (2) were arranged in conditions with good airflow, humidity and in semi-light conditions. The second stage fermentation process was allowed to continue for 4 weeks.

After 4 weeks the sheet material (8) had grown to a thickness of 8 mm. The nutrient medium (4) was drained off and the sheet material (8) was stored in an airtight container.

### EXAMPLE 2

A starter nutrient medium was prepared by adding 4 teabags to 2 litres of boiled water. 160 grams refined white sugar was added to the infusion and stirred, after which the infusion was allowed to cool down for a period of approximately 15 to 20 minutes, thus allowing the infusion temperature to decrease to approximately 20° C. to 25° C. The infusion was introduced into a first-stage fermentation container and a small fungus colony was added to the infusion. In addition, 2 tablespoons of distilled vinegar was added to the infusion to reduce pH of the same.

The liquid was maintained at approximately room temperature for about 4 days. After about 4 days, it was stirred and transferred to a second stage fermentation container, where it was maintained at 23° C. to 28° C. for a further period of about 11 days. After the 11 days, the nutrient medium was drained off and the sheet material harvested and stored.

### EXAMPLE 3

Sheet material formed according Examples 1 and 2 was pulverised to form a gel, after which 1 part of the gel was mixed with 2 parts water. Five airplane tires were set alight. The mixture was sprayed onto the tires and the fires were extinguished in approximately 6 to 8 seconds. There was no subsequent smoke or re-ignition.

### EXAMPLE 4

A car seat was placed on an aircraft runway and two dolls were placed side-by-side on the seat. The one doll and the seat were pre-sprayed with a gel formed of the organic material produced according to Examples 1 and 2, while the other doll was not treated at all. Thereafter, the seat and both dolls were drenched in petrol and set alight. The fire was allowed to burn out. After the fire had burnt out, the seat and the doll, which had been treated, were in perfect condition (even the lace on the doll's dress was undamaged!), while the other doll was almost incinerated.

It will be appreciated that many other embodiments of the invention may be possible without departing from the spirit or scope of the invention as defined in the claims.

1. A method of producing an organic material wherein the material multiplies through a process of germination and wherein the method is characterised therein that it includes a two-stage fermentation process, the method comprising the steps of preparing a starter nutrient medium in which an organic bacterial fungus will grow; adding a starter culture of the organic bacterial fungus to the nutrient medium; permitting the mixture of nutrient medium and fungus culture to undergo a first stage fermentation process; transferring the mixture to a fermentation container; allowing the mixture to undergo a second stage fermentation process until the organic material has germinated fully; and harvesting the organic material.

2. The method as claimed in claim 1 characterised therein that the nutrient medium is an infusion of plant material and water.

3. The method as claimed in claim 2 characterised therein that the nutrient medium is an infusion of tealeaves and water, wherein the tealeaves are selected from a group including, although not limited to, Cyclopia Intermedia, Matricaria Recutita, Aspalathus linearis and/or Lavender.

4. The method as claimed in claim 1 characterised therein that the organic bacterial fungus is a fungus colony of the specific plant material to be infused during preparation of the nutrient medium.

5. The method as claimed in claim 1 characterised therein that the method includes the further step of introducing an acidic medium into the starter nutrient medium for reducing pH of the same.

6. The method as claimed in claim 5 characterised therein that the acidic medium is distilled vinegar.

7. The method as claimed in claim 5 characterised therein that the acidic medium is acidic nutrient medium from a previous fermentation process.

8. The method as claimed in claim 1 characterised therein that the first stage fermentation process occurs for a period of between 3 and 5 days, while the second stage fermentation process occurs for a period of between 10 and 12 days, or until the organic material has grown into a sheet of approximately 8 mm to 10 mm thick.

9. The method as claimed in claim 1 characterised therein that the first stage fermentation process occurs in the absence of direct sunlight.

10. The method as claimed in claim 1 characterised therein that the mixture remains undisturbed during the first stage fermentation process.

11. The method as claimed in claim 1 characterised therein that the nutrient medium is maintained at a temperature ranging between 20° C. and 30° C., and optimally at a temperature range of 23° C.-28° C., during both the first and the second stage fermentation processes.

12. The method as claimed in claim 1 characterised therein that the fermentation container is an elongate and substantially cylindrical container.

13. The method as claimed in claim 1 characterised therein that the fermentation container is a fermentation pipe having a diameter in the order of 100 mm, and a length in the order of 6 m.

14. The method as claimed in claim 1 characterised therein that the mixture of nutrient medium and fungus culture is introduced into the fermentation container such that the surface area of the nutrient medium is below the horizontal centerline of the cylindrical fermentation container, and more particularly, such that the surface area of the nutrient medium is between 8 mm and 10 mm below the horizontal centerline of the cylindrical fermentation container, the arrangement being such that the sheet material is permitted to germinate until it has reached the horizontal centerline of the fermentation container, after which it is harvested, at which point the sheet material should have a thickness of between 8 mm and 10 mm.

15. A method of producing an organic material wherein the material multiplies through a process of germination, the method comprising the steps of preparing a starter nutrient medium in which an organic bacterial fungus will grow; adding an acidic medium to the starter nutrient medium for reducing pH of the same; adding a starter culture of the organic bacterial fungus to the nutrient medium; and permitting the mixture of acidic starter nutrient medium and fungus culture to undergo fermentation until the organic material has germinated fully.

16. The method as claimed in claim 15 characterised therein that the acidic medium is distilled vinegar.

17. The method as claimed in claim 15 characterised therein that the acidic medium is an acidic nutrient medium from a previous fermentation process.

18. The method as claimed in claim 15 characterised therein that the method includes a two-stage fermentation process wherein the mixture of starter nutrient medium and fungus culture is permitted to undergo a first stage fermentation process, after which the mixture is transferred to a fermentation container and allowed to undergo a second stage fermentation process until the organic material has germinated fully.

19. The method as claimed in claims 1 or 15 characterised therein that the method includes the further step of, subsequent to germination and harvesting of the organic sheet material, utilizing the then nutrient medium resulting from the fermentation process as the starter nutrient medium for growing a second organic sheet material, this further step being characterised therein that it does not require addition of a starter culture of the organic bacterial fungus to the nutrient medium.

20. The method as claimed in claim 19 characterised therein that the process of harvesting the organic sheet material and utilizing the then nutrient medium resulting from the fermentation process as the starter nutrient medium for growing another organic sheet material, without the need for adding additional starter culture to the nutrient medium, is repeated a number of times.

21. The method as claimed in claims 1 or 15 characterised therein that the material is dried to form a dry sheet.

22. The method as claimed in claims 1 or 15 characterised therein that the material pulverised to form a gel.

23. The method as claimed in claim 22 characterised therein that the material is mixed with water to form a fire-extinguishing medium and general thermal barrier.

24. Use of an organic material as claimed in claims 1 and 15 as a fire-extinguishing material in commercial fire extinguishers, sprinkler systems for buildings, ships, trains or the like vehicles, in heat protective clothing, for use in extinguishing forest fires or vegetation fires.

25. Use of an organic material as claimed in claims 1 and 15 as a fire-prevention material in buildings, aircraft, ships or the like, in paints, varnishes or the like to be applied to buildings, aircraft, ships or the like, in the manufacturing of roof tiles, dry walling, partitions, ceiling boards, cement formulations, on aircraft runway surfaces to decrease fire risks during emergency landings, and inclusion in furniture upholstery, fabrics, polymers and the like.

26. Use of an organic material as claimed in claims 1 and 15 for extinguishing solvent fires, such as petrol fires by spraying the material onto the fire.

27. Use of an organic material as claimed in claims 1 and 15 in emulsifying oil and water during a process of recovering oil from a source, such as during oil winning from an oil well.

28. Use of an organic material as claimed in claims 1 and 15 for terminating smoke emission by spraying the material over the smoke.

29. A method of producing an organic material substantially as herein illustrated and exemplified with reference to the accompanying drawing and examples.

30. The use of an organic material, produced according to the method of the invention, substantially as herein exemplified with reference to the accompanying examples.
